(19) United States
(12) Patent Application Publication
(10) Pub. No.: US 2015/0030534 A1
(43) Pub. Date: Jan. 29, 2015

(54) ANTIBODY COCKTAILS FOR BREAST CANCER RADIOIMMUNOTHERAPY

(71) Applicant: Rutgers, The State University of New Jersey, New Brunswick, NJ (US)

(72) Inventors: Roger W. Howell, Millington, NJ (US); John M. Akudugu, Bellville (ZA); Jordan B. Pasternack, Morganville, NJ (US)

(21) Appl. No.: 14/444,391

(22) Filed: Jul. 28, 2014

Related U.S. Application Data
(60) Provisional application No. 61/859,060, filed on Jul. 26, 2013.

(51) Int. Cl. A61K 51/10; A61K 45/06

(52) U.S. Cl. CPC A61K 51/1051; A61K 45/06
USPC 424/1.49

(57) ABSTRACT

The present invention provides compositions comprising a therapeutically effective combination of at least two of radionuclide-labeled anti-Epidermal Growth Factor Receptor (anti-EGFR), radionuclide-labeled anti-CD-44, and radionuclide-labeled anti-CD-73. or their respective antigen binding portions, and a pharmaceutically acceptable carrier, and method of use thereof for the treatment of breast cancer.

### CROSS-REFERENCE TO RELATED APPLICATIONS

The present application claims the benefit of priority under 35 U.S.C. 35 §119(e) of U.S. Provisional Application No. 61/859,060 filed on Jul. 26, 2013, the disclosure of which is incorporated by reference herein in it entirety.

### STATEMENT REGARDING FEDERALLY SPONSORED RESEARCH OR DEVELOPMENT

This invention was made with government support under Contract No. 5R25CA019536-32 awarded by the National Institutes of Health. The government has certain rights in the invention.

### BACKGROUND OF THE INVENTION

Radioimmunotherapy (RIT) is a cancer therapy that involves the administration of radionuclide-labeled antibodies to a patient. The antibody is typically chosen based upon antigen expression on the cells comprising the specific type of cancer being treated. The radiolabeled antibody then binds to the antigen present on the cancer cells and delivers a lethal dose of radiation. Despite the promise of RIT, only two agents (BEXXAR®, ¹³¹I-anti-CD20; ZEVALIN®, ⁹⁰Y-anti-CD20) have thus far been approved by the US FDA for therapeutic use. Although complex regulatory issues have played some role in this, the limited number of approved agents is largely due to difficulties in adequately irradiating all of the cancer.

### BRIEF SUMMARY OF THE INVENTION

In one embodiment, the present invention provides a composition comprising a combination of radionuclide-labeled antibodies, or antigen binding portions thereof, and a pharmaceutically acceptable carrier. In one embodiment, the combination of antibodies or antigen binding portions thereof comprises at least two of anti-Epidermal Growth Factor Receptor (anti-EGER), anti-CD-73, and anti-CD-44. Combinations can include antibodies to different epitopes of the same molecule, e.g. antibodies to different epitopes of CD-44.

In another embodiment, the present invention provides a method of treating breast cancer. The method includes the steps of identifying a subject in need of such treatment, and administering to the subject a composition comprising a combination of radionuclide-labeled antibodies, or antigen binding portions thereof, and a pharmaceutically acceptable carrier. In one embodiment, the combination of antibodies or antigen binding portions thereof comprises at least two of anti-Epidermal Growth Factor Receptor (anti-EGER), anti-CD-73, and anti-CD-44. Combinations can include antibodies to different epitopes of the same molecule, e.g. antibodies to different epitopes of CD-44. In another embodiment, the method further comprises the administration of a second therapeutic agent for the prevention or treatment of breast cancer.

### BRIEF DESCRIPTION OF THE DRAWINGS

FIG. 1 shows lognormal uptake of APC-labeled anti-EGFR(AY13) (A), AF-488-labeled anti-CD-44 (691534) (B), Pacific Blue-labeled anti-CD-73(AD2) (C), and PE-labeled anti-CD-44 (BJ18) (D) in MDA-MB-231 cells. All labeling concentrations were 1 μ/ml.

FIG. 2 shows distribution of number of molecules of anti-EGER(AY13) (A), anti-CD-44 (691534) (B), anti-CD-73 (C), and anti-CD-44 (BJ18) (D) hound to MDA-MB-231 cells. The concentration of each antibody in the cocktail was 1 μg/ml.

FIG. 3 shows distribution of uptake of antibodies that target different epitopes of EGFR and CD-44, respectively.

FIG. 4 shows dependence of Monte Carlo-derived surviving fractions on constituents of antibody cocktail and distribution of antibody uptake among targeted cells. Antibodies were labeled with astatine-211. Note increased cell killing afforded by cocktails, and differences in surviving fractions that arise when experimentally measured lognormal distributions are compared with an assumed uniform distribution.

FIG. 5 shows Monte Carlo-derived surviving fraction of MDA-MB-231 cells for select antibody treatments.

FIG. 6 shows dose distributions to MDA-MB-231 cells for different permutations of the antibody cocktail.

### DETAILED DESCRIPTION OF INVENTION

In one embodiment, the present invention provides a composition comprising a combination of radionuclide-labeled antibodies, or antigen binding portions thereof, and to pharmaceutically acceptable carrier. In one embodiment, the combination of antibodies or antigen binding portions thereof comprises at least two of anti-Epidermal Growth Factor Receptor (anti-EGFR), anti-CD-73, and anti-CD-44. Combinations can include antibodies to different epitopes of the same molecule, e.g. antibodies to different epitopes of CD-44.

The term “antibody” (Ab) as used herein includes monoclonal antibodies, polyclonal antibodies, multispecific antibodies (for example, bispecific antibodies and polyreactive antibodies), and antibody fragments. Thus, the term “antibody” as used in any context within this specification is meant to include, but not be limited to, any specific binding member, immunoglobulin class and/or isotype (e.g., IgG1, IgG2, IgG3, IgG4, IgM, IgA, IgE and IgM); and biologically relevant fragment or specific binding member thereof, including but not to limited to Fab, F(ab′)2, Fv, and scFv (single chain or related entity). It is understood in the art that an antibody is a glycoprotein having at least two heavy (H) chains and two light (L) chains inter-connected by disulfide bonds, or an antigen binding portion thereof. A heavy chain is comprised of a heavy chain variable region (VH) and a heavy chain constant region (CH1, CH2 and CH3). A light chain is comprised of a light chain variable region (VL) and a light chain constant region (CL). The variable regions of both the heavy and light chains comprise framework regions (FWR) and complementarity determining regions (CDR). The four FWR regions are relatively conserved while CDR regions (CDR1, CDR2 and CDR3) represent hypervariable regions and are arranged from NH2 terminus to the COOH terminus as follows: FWR1, CDR1, FWR2, CDR2, FWR3, CDR3, and FWR4. The variable regions of the heavy and light chains contain a binding domain that interacts with an antigen while, depending of the isotype, the constant region(s) may mediate the binding of the immunoglobulin to host tissues or factors.

Also included in the definition of “antibody” as used herein are chimeric antibodies, humanized antibodies, and recombinant antibodies, human antibodies generated from a transgenic non-human animal, as well as antibodies selected from libraries using enrichment technologies available to the artisan.

The term “variable” refers to the fact that certain segments of the variable (V) domains differ extensively in sequence among antibodies. The V domain mediates antigen binding and defines specificity of a particular antibody for its particular antigen. However, the variability is not evenly distributed across the 110-amino acid span of the variable regions. Instead, the V regions consist of relatively invariant stretches called framework regions (FRs) of 15-30 amino acids separated by shorter regions of extreme variability called “hypervariable regions” that are each 9-12 amino acids long. The variable regions of native heavy and light chains each comprise four FRs, largely adopting a beta sheet configuration, connected by three hypervariable regions, which form loops connecting, and in some cases forming part of, the beta sheet structure. The hypervariable regions in each chain are held together in close proximity by the FRs and, with the hypervariable regions from the other chain, contribute to the formation of the antigen-binding site of antibodies (see, for example, Kabat et al., Sequences of Proteins of Immunological Interest, 5th Ed. Public Health Service, National institutes of Health, Bethesda, Md. (1991)).

The term “hypervariable region” as used herein refers to the amino acid residues of an antibody that are responsible for antigen binding. The hypervariable region generally comprises amino acid residues from a “complementarity determining region” (“CDR”).

The term “monoclonal antibody” as used herein refers to an antibody obtained from a population of substantially homogeneous antibodies, i.e., the individual antibodies comprising the population are identical except for possible naturally occurring mutations that may be present in minor amounts. The term “polyclonal antibody” refers to preparations that include different antibodies directed against different determinants (“epitopes”).

The monoclonal antibodies herein include “chimeric” antibodies in which a portion of the heavy and/or light chain is identical with, or homologous to, corresponding sequences in antibodies derived from a particular species or belonging to a particular antibody class or subclass, while the remainder of the chain(s) is identical with, or homologous to, corresponding sequences in antibodies derived from another species or belonging to another antibody class or subclass, as well as fragments of such antibodies, so long as they exhibit the desired biological activity (see, for example. U.S. Pat. No. 4,816,567; and Morrison et al., Proc. Natl. Acad. Sci. USA, 81:6851-6855 (1984)). Chimeric antibodies included herein include antibodies having one or more human antigen binding sequences (for example, CDRs) and containing one or more sequences derived from a non-human antibody, for example, an FR or C region sequence. In addition, chimeric antibodies included herein are those comprising a human variable region antigen binding sequence of one antibody class or subclass and another sequence, for example, FR or C region sequence, derived from another antibody class or subclass.

A “humanized antibody” generally is considered to be a human antibody that has one or more amino acid residues introduced into it from a source that is non-human. These non-human amino acid residues often are referred to as “import” residues, which typically are taken from an “import” variable region. Humanization may be performed following the method of Winter and co-workers (see, for example, Jones et al., Nature, 321:522-525 (1986); Reichmann et al., Nature, 332:323-327 (1988); Verhoeyen et al., Science, 239:1534-1536 (1988)), by substituting import hypervariable region sequences for the corresponding sequences of a human antibody. Accordingly, such “humanized” antibodies are chimeric antibodies (see, for example, U.S. Pat. No. 4,816,567), where substantially less than an intact human variable region has been substituted by the corresponding sequence from a non-human species.

An “antibody fragment” comprises a portion of an intact antibody, such as the antigen binding or variable region of the intact antibody. Examples of antibody fragments include, but are not limited to, Fab, Fab′, F(ab′)2, and Fv fragments; diabodies; linear antibodies (see, for example, U.S. Pat. No. 5,641,870; Zapata et al., Protein Eng. 8(10): 1057-1062 [1995]); single-chain antibody molecules; and multispecific antibodies formed from antibody fragments. “Fv” is the minimum antibody fragment that contains a complete antigen-recognition and antigen-binding site. This fragment contains a dimer of one heavy- and one light-chain variable region domain in tight, non-covalent association. From the folding of these two domains emanate six hypervariable loops (three loops each from the H and L chain) that contribute the amino acid residues for antigen binding and confer antigen binding specificity to the antibody. However, even a single variable region (or half of an Fv comprising only three CDRs specific for an antigen) has the ability to recognize and bind antigen, although at a lower affinity than the entire binding site.

“Single-chain Fv” (“sFv” or “scFv”) are antibody fragments that comprise the VH and VL antibody domains connected into a single polypeptide chain. The sFv polypeptide can further comprise a polypeptide linker between the VH and VL domains that enables the sFv to form the desired structure for antigen binding. For a review of sFv, see, for example, Pluckthun in The Pharmacology of Monoclonal Antibodies, vol. 113. Rosenburg and Moore eds., Springer-Verlag, New York, pp. 269-315 (1994); Borrebaeck 1995, infra.

The term “diabodies” refers to small antibody fragments prepared by constructing sFv fragments with short linkers (about 5-10 residues) between the VH and VL domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the VH and VL domains of the two antibodies are present on different polypeptide chains. Diabodies are described more fully in, for example, EP 404,097; WO 93/11161; and Hollinger et al., Proc. Natl. Acad. Sci, USA, 90:6444-6448 (1993). Domain antibodies (dAbs), which can be produced in fully human form, are the smallest known antigen-binding fragments of antibodies, ranging from about 11 kDa to about 15 kDa. DAbs are the robust variable regions of the heavy and light chains of immunoglobulins (VU and VL, respectively). They are highly expressed in microbial cell culture, show favorable biophysical properties including, for example, but not limited to, solubility and temperature stability, and are well suited to selection and affinity maturation by in vitro selection systems such as, for example, phage display. DAbs are bioactive as monomers and, owing to their small size and inherent stability, can be formatted into larger molecules to create drugs with prolonged serum half-lives or other pharmacological activities. Examples of this technology have been described in, for example, WO9425591 for antibodies derived from Camelidae heavy chain Ig, as well in US20030130496 describing the isolation of single domain fully human antibodies from phage libraries. Fv and sFv are the only species with intact combining sites that are devoid of constant regions. sFv fusion proteins can be constructed to yield fusion of an effector protein at either the amino or the carboxy terminus of an sFv. See, for example, Antibody Engineering, ed. Borrebaeck, supra. The antibody fragment also can be a “linear antibody”, for example, as described in U.S. Pat. No. 5,641,870 for example. Such linear antibody fragments can be monospecific or bispecific.

Typically, the antibodies of the described invention are produced recombinantly, using vectors and methods available in the art. Human antibodies also can be generated by in vitro activated B cells (see, for example, U.S. Pat. Nos. 5,567,610 and 5,229,275). General methods in molecular genetics and genetic engineering useful in the present invention are described in the current editions of Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc,); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987, Liss, Inc. New York, N.Y.), and Gene Transfer and Expression Protocols, pp. 109-128, ed. E. J. Murray, The Humana Press Inc., Clifton, N.J.). Reagents, cloning vectors, and kits for genetic manipulation are available from commercial vendors such as BioRad, Stratagene, Invitrogen, ClonTech and Sigma-Aldrich Co.

Human antibodies also can be produced in transgenic animals (for example, mice) that are capable of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice results in the production of human antibodies upon antigen challenge. See, for example, Jakobovits et al., Proc. Natl. Acad. Sci. USA. 90:2551 (1993); Jakobovits et al., Nature, 362:255-258 (1993); Bruggemann et al., Year in Immuno., 7:33 (1993); U.S. Pat. Nos. 5,545.806, 5,569,825, 5,591,669 (all of GenPharm); U.S. Pat. No. 5,545,807; and WO 97/17852

Various techniques have been developed for the production of antibody fragments. Traditionally, these fragments were derived via proteolytic digestion of intact antibodies (see, for example, Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Fab′-SH fragments can be directly recovered from E. coli and chemically coupled to form F(ab′)2 fragments (see, for example. Carter et al., Bio/Technology 10:163-167 (1992)). According to another approach, F(ab′)2 fragments can be isolated directly from recombinant host cell culture. Fab and F(ab′)2 fragment with increased in vivo half-life comprising a salvage receptor binding epitope residues are described in U.S. Pat. No. 5,869,046. Other techniques for the production of antibody fragments will be apparent to the skilled practitioner.

Other techniques that are known in the art for the selection of antibody fragments from libraries using enrichment technologies, including but not limited to phage display, ribosome display (Hanes and Pluckthun, 1997, Proc. Nat. Acad. Sci. 94: 4937-4942), bacterial display (Georgiou, et al., 1997, Nature Biotechnology 15: 29-34) and/or yeast display (Kieke, et al., 1997, Protein Engineering 10: 1303-1310) may be utilized as alternatives to previously discussed technologies to select single chain antibodies. Single-chain antibodies are selected from a library of single chain antibodies produced directly utilizing filamentous phage technology. Phage display technology is known in the art (e.g., see technology from Cambridge Antibody Technology (CAT)) as disclosed in U.S. Pat. Nos. 5,565,332; 5,733,743; 5,871,907; 5,872,215; 5,885,793; 5,962,255; 6,140,471; 6,225,447; 6,291,650; 6,492,160; 6,521,404; 6,544,731; 6,555,313; 6,582,915; 6,593,081; see also Vaughn, et al. 1996, Nature Biotechnology 14: 309-314). Single chain antibodies may also be designed and constructed using available recombinant DNA technology, such as a DNA amplification method (e.g., PCR), or by using a respective hybridoma cDNA as a template.

Antibodies against Epithelial Cell Adhesion Molecule, Epidermal Growth Factor Receptor, and Tag-72 (a high molecular weight glycoprotein present in human adenocarcinoma) are known in the art and commercially available, and can also be generated by methods known in the art, for example as described above.

The antibodies or antigen binding portions thereof in the compositions of the present invention are conjugated to a radionuclide. The radionuclide may be attached directly to an antibody or antigen binding portion thereof to form an immunoconjugate. Immunoconjugates may be formed by direct covalent attachment of the radionuclide to a functional group on the antibody, or the radionuclide may be conjugated to a chelating moiety that is attached to the antibody or fragment thereof. Methods for conjugating radionuclides to antibodies are known to those of skill in the art.

The radionuclide may be any radionuclide useful for RIT including, for example, ¹¹C, ¹³N, ¹⁵O, ³²P, ³³P, ⁴⁷C, ⁵¹Cr, ⁵⁷Co, ⁵⁸Co, ⁵⁹Fe, ⁶²Cu, ⁶⁷Cu, ⁶⁷Ga, ⁷⁵Br, ⁷⁵Se, ⁷⁵Se, ⁷⁶Br, ⁷⁷As, ⁷⁷Br, ^{80m}Br, ⁸⁹Sr, ⁹⁰Y, ⁹⁵Ru, ⁹⁷Ru, ⁹⁹M, ^{99m}Tc, ^{103m}Rh, ¹⁰³Ru, ¹⁰⁵Rh, ¹⁰⁵Ru, ¹⁰⁷Hg, ¹⁰⁹Pd, ¹⁰⁹Pt, ¹¹¹Ag, ¹¹¹In, ^{113m}In, ¹¹⁹Sb, ^{121m}Te, ^{122m}Te, ¹²⁵I, ^{125m}Te, ¹²⁶I, ¹³¹I, ¹³³I, ¹⁴²Pr, ¹⁴³Pr, ¹⁴⁹Pm, ¹⁵²Dy, ¹⁵³Sm, ¹⁶¹Ho, ¹⁶¹Tb, ¹⁶⁵Tm, ¹⁶⁶Dy, ¹⁶⁶Ho, ¹⁶⁷Tm, ¹⁶⁸Tm, ¹⁶⁹Er, ¹⁶⁹Yb, ¹⁷⁷Lu, ¹⁸⁶Re, ¹⁸⁸Re, ^{189m}Os, ¹⁸⁹Re, ¹⁹²Ir, ¹⁹⁴Ir, ¹⁹⁷Pt, ¹⁹⁸Au, ¹⁹⁹Au, ¹⁹⁹Au, ²⁰¹Tl, ²⁰³Hg, ²¹¹At, ²¹¹Bi, ²¹¹Pb, ²¹²Bi, ²¹²Pb, ²¹³Bi, ²¹⁵Pb, ²¹⁷At, ²¹⁹Rn, ²²¹Fr, ²²³Ra, ²²⁴Ac, ²²⁵Ac and ²⁵⁵Fm. In a preferred embodiment, the radionuclide is ¹³¹I, ⁹⁰Y, ¹¹¹In or ^{99m}Tc.

The compositions of the invention may comprise a therapeutically effective combination of at least two of radionuclide-labeled anti-EGFR (AY13), radionuclide-labeled anti-CD-73 (AD2), radionuclide labeled anti-CD-44 (691534), and radionuclide labeled anti-CD-44 (BJ18), or their respective antigen binding portions, and a pharmaceutically acceptable carrier. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not hunted to, biocompatibie vehicles, adjuvants, additives, and diluents to achieve a composition usable as a dosage form. Examples of other carriers include colloidal silicon oxide, magnesium stearate, cellulose, and sodium lauryl sulfate.

The above-described composition, in any of the forms described above, can be used for treating a cellular proliferative disorder such as breast cancer. Accordingly, in another embodiment, the present invention provides a method of treating breast cancer. The method includes the steps of identifying a subject in need of such treatment, and administering to the subject a composition comprising a therapeutically effective combination of at least two of radionuclide-labeled anti-EGFR, radionuclide-labeled anti-CD-73, and radionuclide labeled anti-CD-44, or their respective antigen binding portions, and a pharmaceutically acceptable carrier.

An effective amount refers to the amount of a combination of radionuclide antibodies or portions thereof that is required to confer a therapeutic effect on a treated subject. Effective doses will vary, as recognized by those skilled in the art, depending on the types of conditions treated, route of administration, excipient usage, and the possibility of co-usage with other therapeutic treatment.

A pharmaceutical composition of this invention can be administered to a subject parenterally, orally, nasally, rectally, topically, or buccally. The term “parenteral” as used herein refers to, but not limited to, subcutaneous, intracutaneous, intravenous, intrmuscular, intraarticular, or intraarterial injection, as well as any suitable infusion technique. A sterile injectable composition can be a solution or suspension in a non-toxic parenterally acceptable diluent or solvent. Such solutions include, but are not limited to, 1,3-butanediol, mannitol, water, Ringer's solution, saline, phosphate buffer solution, and isotonic sodium chloride solution. In addition, fixed oils are conventionally employed as a solvent or suspending medium (e.g., synthetic mono- or di-glycerides). Fatty acids, such as, but not limited to, oleic acid and its glyceride derivatives, are useful in the preparation of injectables, as are natural pharmaceutically acceptable oils, such as, but not limited to, olive oil or castor oil, polyoxyethylated versions thereof. These oil solutions or suspensions also can contain a long chain alcohol diluent or dispersant such as, but not limited to, carboxymethyl cellulose, or similar dispersing agents. Other commonly used surfactants, such as, but not limited to, TWEENS or SPANS or other similar emulsifying agents or bioavailability enhancers, which are commonly used in the manufacture of pharmaceutically acceptable solid, liquid, or other dosage forms also can be used for the purpose of formulation.

As used herein, a “subject” refers to a human and a non-human animal. Examples of a non-human animal include all vertebrates, e.g., mammals, such as non-human mammals, non-human primates (particularly higher primates), dog, rodent (e.g., mouse or rat), guinea pig, cat, and rabbit, and non-mammals, such as birds, amphibians, reptiles, etc. In one embodiment, the subject is a human. In another embodiment, the subject is an experimental, non-human animal or animal suitable as a disease model. “Treating” or “treatment” refers to administration of a compound or agent to a subject who has a disorder with the purpose to cure, alleviate, relieve, remedy, delay the onset of, prevent, or ameliorate the disorder, the symptom of the disorder, the disease state secondary to the disorder, or the predisposition toward the disorder. An “effective amount” or “therapeutically effective amount” refers to an amount of the compound or agent that is capable of producing a medically desirable result in a treated subject. The treatment method can be performed in vivo or ex vivo, alone or in conjunction with other drugs or therapy. A therapeutically effective amount can be administered in one or more administrations, applications or dosages and is not intended to be limited to a particular formulation or administration route.

In another embodiment, the method further comprises the administration of a second therapeutic agent for the prevention or treatment of breast cancer. The composition of the invention and the second therapeutic agent may be administered simultaneously or sequentially. For sequential treatment, the second therapeutic agent may be administered before or after the composition of the invention. Therapeutic agents for the treatment of breast cancer are known in the art and include, for example, chemotherapeutic agents such as cyclophosphamide, doxorubicin, navelbine, capecitabine, paxlitaxel, mitomycin C, carboplatin, daunorubicin, epirubicin, fluorouracil, gemcitabine, eribulin, ivabepilone, methotrexate, mutamycin, mitoxantrone, vinorelbine, docetaxel, thiotepa, vincristine and capecitabine; hormonal agents such as aromatase inhibitors, selective estrogen receptor modulators (SERMs) and estrogen receptor downregulators (ERDs); and targeted therapies such as trastuzumab, lapatinib, bevacizumab, pertuzumab and everolimus.

The following non-limiting example serves to further illustrate the present invention.

### EXAMPLE

This example illustrates a method to increase the effectiveness of RIT by formulating cocktails of radiolabeled antibodies. Four monoclonal antibodies (Ab) were pre-labeled with fluorochromes, and breast cancer cells were treated with graded concentrations. Optimal concentrations were determined by flow cytometry and Monte Carlo analysis. Cells were treated with Ab cocktails and it was found that the overall distribution of Ab binding was changed, resulting in a predicted increase in killing of the tumor cell population.

### Materials and Methods Cell Culture

Cell culture protocol followed Akudugu et al. (Akudugu J M, Howell R W. Flow cytometry-assisted Monte Carlo simulation predicts clonogenic survival of cell populations with lognormal distributions of radiopharmaceuticals and anticancer drugs. Int J Radiat Biol. 2011; 88:286-293; Akudugu J M, Neti P V S V, Howell R W. Changes in lognormal shape parameter guide design of patient-specific radiochemotherapy cocktails. J Nucl Med. 2011; 52:642-649.).

MDA-MB-231 human breast cancer cells were grown in minimum essential medium (MEM). The medium was supplemented with 10% fetal calf serum, 2 mM L-glutamine, 100 units/ml penicillin, 100 streptomycin, 10 mg/ml non-essential amino acids. Cells were grown as monolayers in 175-cm² flasks (37° C., 5% CO₂-95% air, humidified) and were used fir experiments (passages 29-34) at 70-90% confluence.

### Antibody Labeling

Cells were trypsinized (0.5% TrypsintEDTA), resuspended in medium, syringed using a 21 gauge needle, counted, and then centrifuged for 5 min. Cells were then resuspended in medium containing only penicillin-streptomycin and 3 ml containing 10⁶ cells was placed in 5 ml round bottom tubes. All tubes were centrifuged for 5 min. The cells were then resuspended in 100 μl of 1 μg/ml antibody solution. Biolegend APC anti-EGFR(AY13), Pacific Blue anti CD-73(AD2), PE anti CD-44(BJ18), PE anti-CD-221(1H7). Pacific. Blue anti-EpCAM(9C4), R&D Systems AF-488 anti CD-44(691534), AF-700 anti-GLUT1(202915), Santa Cruz PE anti-EGFR(528), AF-405 anti-EGFR(R-1), PE anti-Tag-72(B72.3), and Millipore PerCP anti-EGFR(LA22) were used. The tubes were then placed on a hematology mixer for 2 h at 37° C., 5% CO₂, 95% air. After the labeling, the cells were washed and resuspended in 1.0 ml of PBS.

### Analysis

The fluorescence intensity of a cell is directly proportional to the amount of bound antibody. Bangs Quantum Simply Cellular Calibration Beads were used to ascertain the number of antibodies bound to each cell using the cell's fluorescence. Cellular uptake of a hypothetical radionuclide is proportional to the uptake of the antibody. With this assumption, the Monte Carlo simulation developed by Akudugu et al. was utilized to predict the surviving fraction of the cells for each antibody treatment for each experiment. These simulations were performed in Excel, where number of antibodies per cell was determined using each cell's fluorescence value. The antibody was then labeled with a radionuclide that emits cytotoxic radiation and the radiation absorbed dose was calculated for each cell. The algorithm was coded in such a way that a user can enter the cellular radiation dose characteristics of a desired radionuclide, and determine cell survival from the resulting cellular dose distribution delivered by a given antibody treatment. Astatine-211, a highly radiotoxic alpha-particle emitter, was used arbitrarily as the radionuclide for all analyses. A modified Monte Carlo simulation was applied to the different antibody treatments to determine the surviving fraction of cells. Surviving fractions of the different antibody treatments were calculated two ways: one in which a lognormal distribution of antibody uptake was assumed, and one in which a uniform distribution of antibody was assumed.

### Results

Preliminary testing was performed to determine the expression of different antigens/epitopes on the MDA-MB-231 cells used. Antibodies that demonstrated poor uptake were eliminated from further study. Based on these findings, an antibody cocktail of four antibodies was prepared: anti-EGFR (AY13), anti-CD-44 (691534), anti-CD-73(AD2), and anti-CD-44 (BJ18).

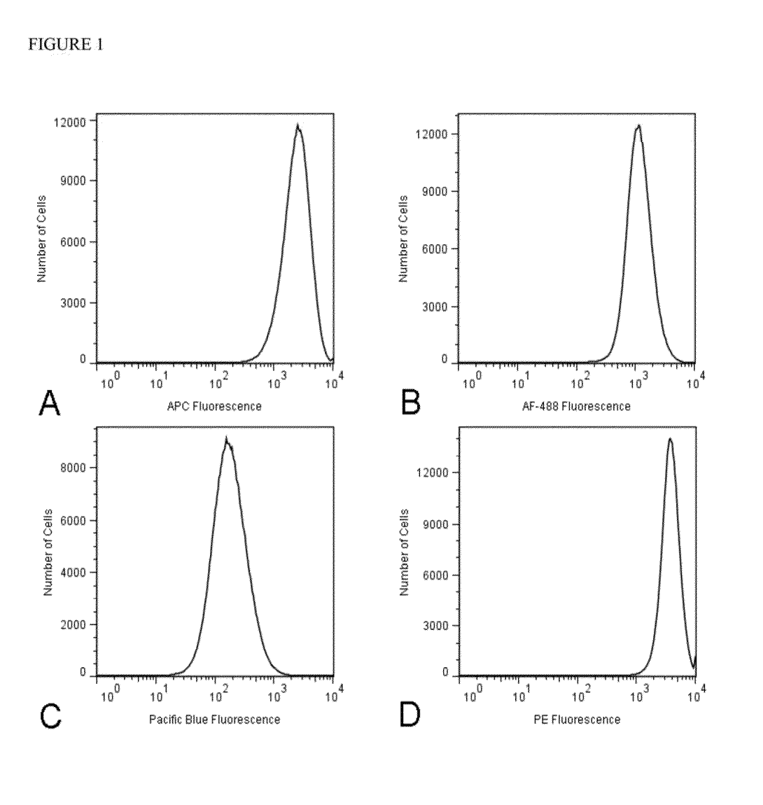

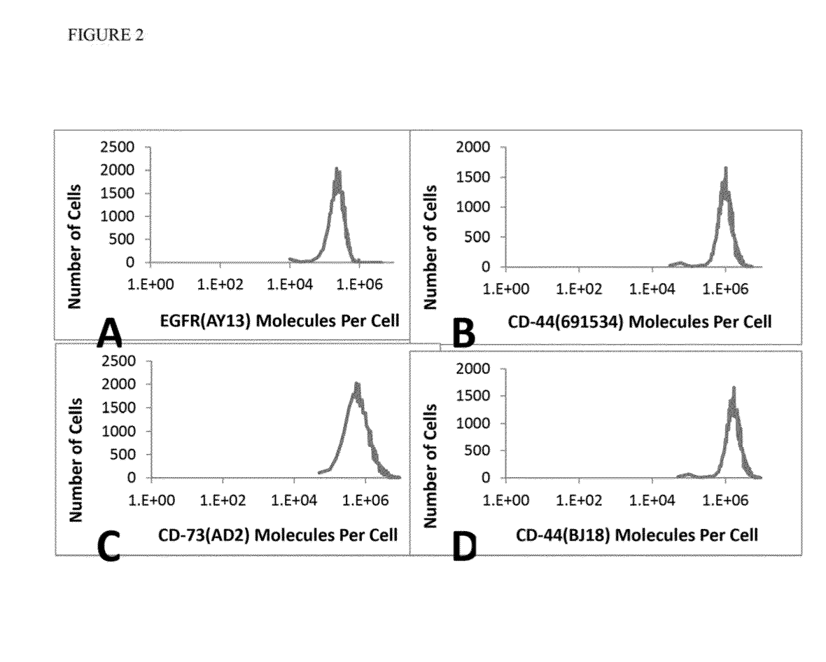

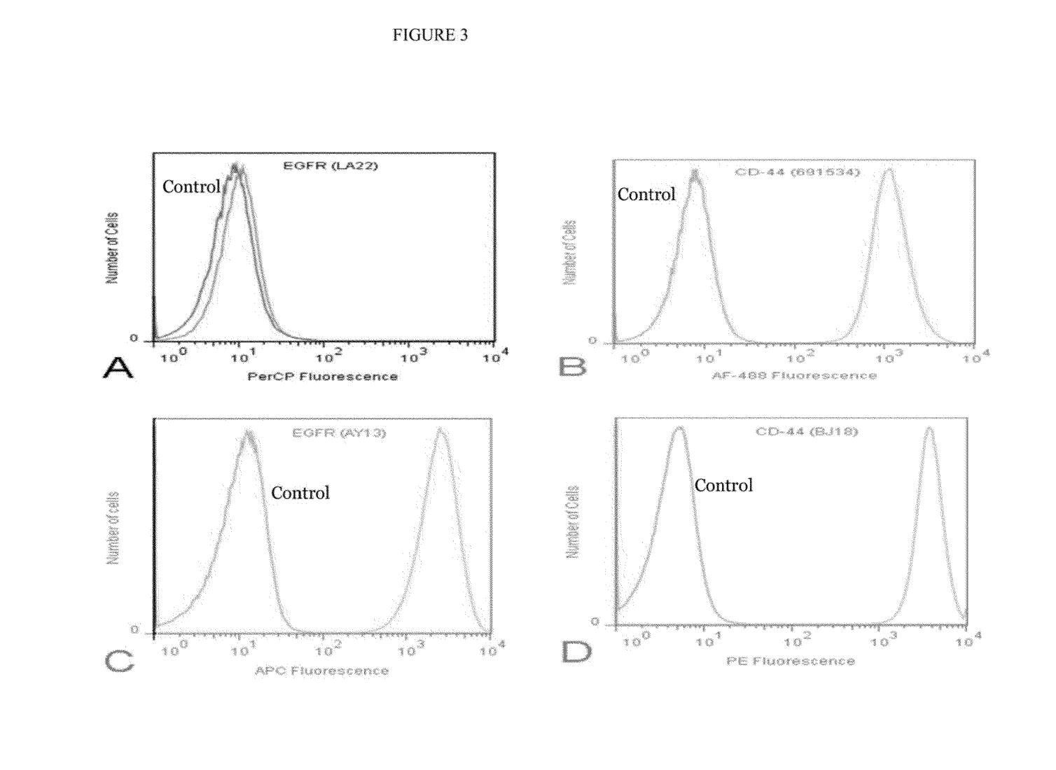

Fluorescence histograms from the four individual antibodies used in the cocktail are shown in FIG. 1. Plotted on semi-log axes, it appears that uptake of each antibody by the MDA-MB-231 cells is reasonably lognormal. While antibody uptake for a given antibody is directly proportional to fluorescence, relative fluorescence values between the different fluorochromes on the four antibodies do not represent relative number of antibodies bound to the cells. This was solved with Bangs calibration beads. FIG. 2 shows the distribution of each antibody on the cells, following calibration. The two epitopes of anti-CD-44 were additive, such that double the antibody was bound to cells when both epitopes were included as opposed to only a single epitope. Combining different epitopes of the same antibody was met with mixed results (FIG. 3). The cells did not appreciably express the LA22 epitope of EGFR (FIG. 3A), but expression of the AY13 epitope of EGFR was profound (FIGS. 2A and 3C). However, both the 691534 (FIGS. 2B and 3B) and BJ18 (FIGS. 2D and 30) epitopes of CD-44 were highly expressed in the cells.

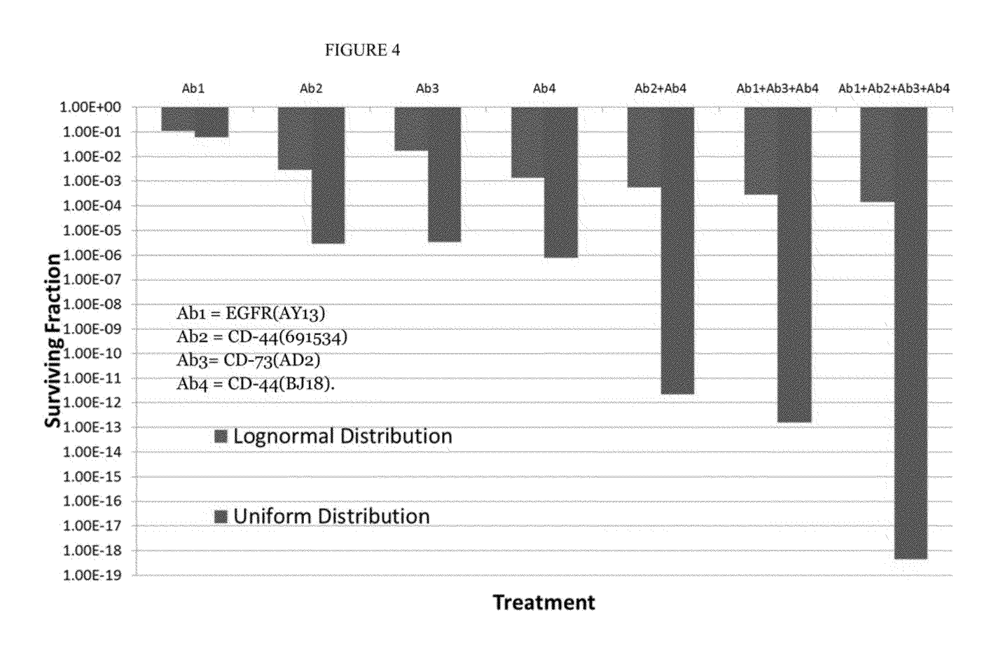

Results for selected permutations of the antibody cocktail are shown in FIG. 4. The surviving fractions that arise when it is assumed that each radiolabeled antibody is uniformly distributed among the targeted population are lower than those when the experimentally measured lognormal distribution is used.

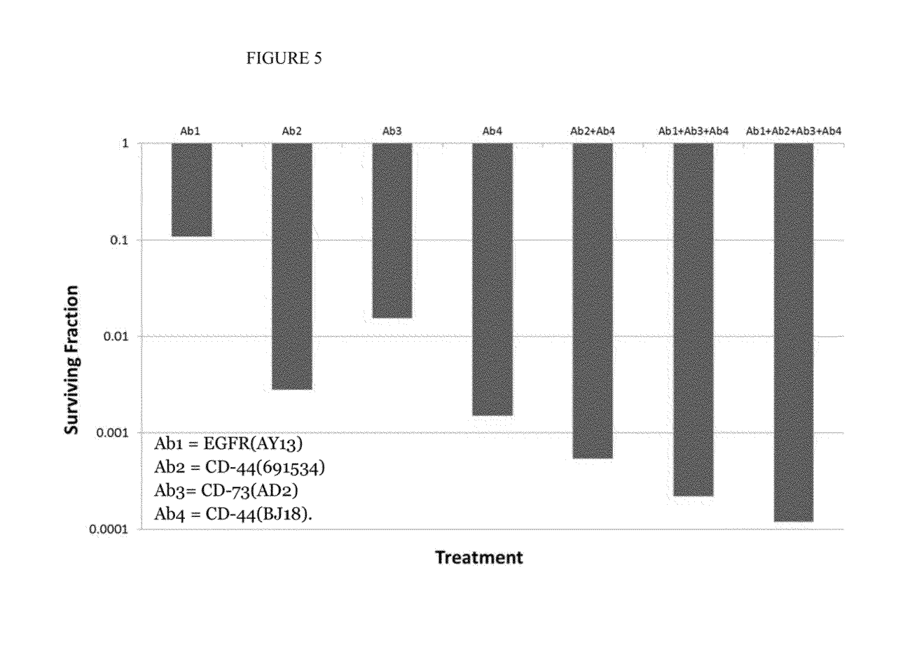

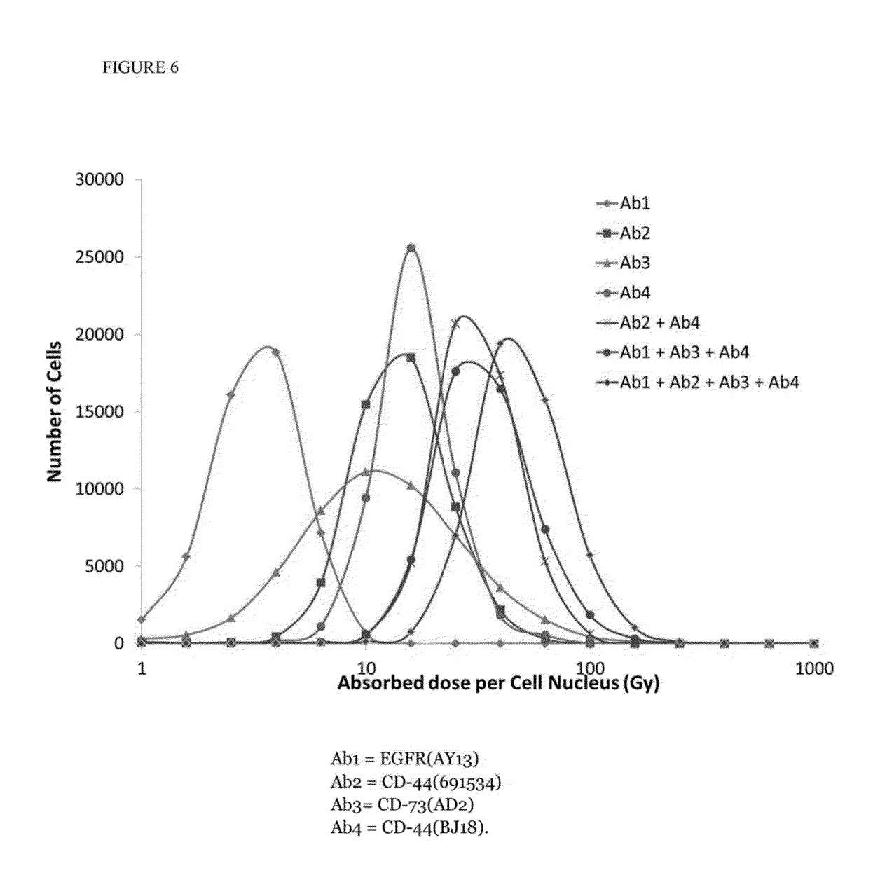

Using the Monte Carlo simulation, a surviving fraction for each permutation of the quadruple antibody cocktail was calculated based upon the absorbed dose to each cell's nucleus. Selected permutations were compared to determine whether or not there is a benefit to using more than one antibody in a RIT treatment. When the best individual antibody is compared with the quadruple cocktail, over a log of kill is gained (FIG. 5). Even adding a single antibody to the best individual antibody results in increased cell killing. To determine whether or not this additional killing is a result of a change in the absorbed dose distribution in the combined treatments, histograms of absorbed dose to the cells treated with different permutations of the cocktail were plotted (FIG. 6). Adding additional antibodies changed the distribution of uptake in the cell population, resulting in up to a several-fold increase in the absorbed dose to the least-irradiated cells when compared to the absorbed doses obtained with the best single antibody treatment.

All references cited herein are incorporated herein by reference in their entireties.

What is claimed is:

1. A composition comprising at least two of radionuclide-labeled anti-Epidermal Growth Factor Receptor (anti-EGFR) or an antigen binding portion thereof, radionuclide-labeled anti-CD-44 or an antigen binding portion thereof, and radionuclide-labeled anti-CD-73 or an antigen binding portion thereof, and a pharmaceutically acceptable carrier.

2. The composition of claim 1 wherein the radionuclides are the same or different and are selected from the group consisting of ¹¹C, ¹³N, ¹⁵O, ³²P, ³³P, ⁴⁷C, ⁵¹Cr, ⁵⁷Co, ⁵⁸Co, ⁵⁹Fe, ⁶²Cu, ⁶⁷Cu, ⁶⁷Ga, ⁶⁷Ga, ⁷⁵Br, ⁷⁵Se, ⁷⁵Se, ⁷⁶Br ⁷⁷As, ⁷⁷Br, ^{80m}Br, ⁸⁹Sr, ⁹⁰Y, ⁹⁵Ru, ⁹⁷Ru, ⁹⁹Mo, ^{99m}Tc, ^{103m}Rh, ¹⁰³Ru, ¹⁰⁵Rh, ¹⁰⁵Ru, ¹⁰⁷Hg, ¹⁰⁹Pd, ¹⁰⁹Pt, ¹¹¹Ag, ¹¹¹In, ^{113m}In, ¹¹⁹Sb, ^{121m}Te, ^{122m}Te, ¹²⁵I, ^{125m}Te, ¹²⁶I, ¹³¹I, ¹³³I, ¹⁴²Pr, ¹⁴³Pr, ¹⁴⁹Pm, ¹⁵²Dy, ¹⁵³Sm, ¹⁶¹Ho, ¹⁶¹Tb, ¹⁶⁵Tm, ¹⁶⁶Dy, ¹⁶⁶Ho, ¹⁶⁷Tm, ¹⁶⁸Tm, ¹⁶⁹Er, ¹⁶⁹Yb, ¹⁷⁷Lu, ¹⁸⁶Re, ¹⁸⁸Re, ^{189m}Os, ¹⁸⁹Re, ¹⁹²Ir, ¹⁹⁴Ir, ¹⁹⁷Pt, ¹⁹⁸Au, ¹⁹⁹Au, ²⁰¹Tl, ²⁰³Hg, ²¹¹At, ²¹¹Bi, ²¹¹Pb, ²¹²Bi, ²¹²Pb, ²¹³Bi, ²¹⁵Pb, ²¹⁷At, ²¹⁹Rn, ²²¹Fr, ²²³Ra, ²²⁴Ac, ²²⁵Ac and ²⁵⁵Fm.

3. The composition of claim 1 wherein the radionuclides are the same or different and are selected from the group consisting of ¹³¹I, ⁹⁰Y, ¹¹¹In or ^{99m}Tc.

4. The method of treating breast cancer in a subject comprising the steps of identifying a subject in need of such treatment, and administering to the subject a composition comprising a therapeutically effective combination of at least two of radionuclide-labeled anti-Epidermal Growth Factor Receptor (anti-EGFR) or an antigen binding portion thereof, radionuclide-labeled anti-CD-44 or an antigen binding portion thereof, and radionuclide-labeled anti-CD-73 or an antigen binding portion thereof, and a pharmaceutically acceptable carrier.

5. The method of claim 4 wherein the radionuclides are the same or different and are selected from the group consisting of ¹¹C, ¹³N, ¹⁵O, ³²P, ³³P, ⁴⁷C, ⁵¹Cr, ⁵⁷Co, ⁵⁸Co, ⁵⁹Fe, ⁶²Cu, ⁶⁷Cu, ⁶⁷Ga, ⁶⁷Ga, ⁷⁵Br, ⁷⁵Se, ⁷⁵Se, ⁷⁶Br ⁷⁷As, ⁷⁷Br, ^{80m}Br, ⁸⁹Sr, ⁹⁰Y, ⁹⁵Ru, ⁹⁷Ru, ⁹⁹Mo, ^{99m}Tc, ^{103m}Rh, ¹⁰³Ru, ¹⁰⁵Rh, ¹⁰⁵Ru, ¹⁰⁷G, ¹⁰⁹Pd, ¹⁰⁹Pt, ¹¹¹Ag, ¹¹¹In, ^{113m}In, ¹¹⁹Sb, ^{121m}Te, ^{122m}Te, ¹²⁵I, ^{125m}Te, ¹²⁶I, ¹³¹I, ¹³³I, ¹⁴²Pr, ¹⁴³Pr, ¹⁴⁹Pm, ¹⁵²Dy, ¹⁵³Sm, ¹⁶¹Ho, ¹⁶¹Tb, ¹⁶⁵Tm, ¹⁶⁶Dy, ¹⁶⁶Ho, ¹⁶⁷Tm, ¹⁶⁸Tm, ¹⁶⁹Er, ¹⁶⁹Yb, ¹⁷⁷Lu, ¹⁸⁶Re, ¹⁸⁸Re, ^{189m}Os, ¹⁸⁹Re, ¹⁹²Ir, ¹⁹⁴Ir, ¹⁹⁷Pt, ¹⁹⁸Au, ¹⁹⁹Au, ²⁰¹Tl, ²⁰³Hg, ²¹¹At, ²¹¹Bi, ²¹¹Pb, ²¹²Bi, ²¹²Pb, ²¹³Bi, ²¹⁵Pb, ²¹⁷At, ²¹⁹Rn, ²²¹Fr, ²²³Ra, ²²⁴Ac, ²²⁵Ac and ²⁵⁵Fm.

6. The method of claim 4 wherein the radionuclides are the same or different and are selected from the group consisting of ¹³¹I, ⁹⁰Y, ¹¹¹In or ^{99m}Tc.

7. The method of claim 4 wherein the subject is a mammal.

8. The method of claim 4 wherein the subject is a human.

9. The method of claim 4 further comprising the step of administering a second therapeutic agent for the prevention or treatment of breast cancer.

10. The method of claim 9 wherein the second therapeutic agent and the composition are administered simultaneously or sequentially.

11. The method of claim 9 wherein the second therapeutic agent is administered before the composition.

12. The method of claim 9 wherein the second therapeutic agent is administered after the composition.

13. The method of claim 9 wherein the second therapeutic agent is a chemotherapeutic agent, a hormonal agent, or a targeted therapeutic agent.

14. The method of claim 13 wherein the chemotherapeutic agent is selected from the group consisting of cyclophosphamide, doxorubicin, navelbine, capecitabine, paxlitaxel, mitomycin C, carboplatin, daunorubicin, epirubicin, fluorouracil, gemcitabine, eribulin, ivabepilone, methotrexate, mutamycin, mitoxantrone, vinorelbine, docetaxel, thiotepa, vincristine and capecitabine.

15. The method of claim 13 wherein the hormonal agents is selected from the group consisting of aromatase inhibitors, selective estrogen receptor modulators (SERMs) and estrogen receptor downregulators (ERDs).

16. The method of claim 13 wherein the targeted therapeutic agent is selected from the group consisting of trastuzumab, lapatinib, bevacizumab, pertuzumab and everolimus.
